(19) United States
(12) Patent Application Publication
(10) Pub. No.: US 2017/0055840 A1
(43) Pub. Date: Mar. 2, 2017

(54) MEASUREMENT PROBE AND OPTICAL MEASUREMENT SYSTEM

(71) Applicant: OLYMPUS CORPORATION, Tokyo (JP)

(72) Inventor: Seiki TORIYAMA, Hino-shi (JP)

(73) Assignee: OLYMPUS CORPORATION, Tokyo (JP)

(21) Appl. No.: 15/244,127

(22) Filed: Aug. 23, 2016

Related U.S. Application Data
(60) Provisional application No. 62/213,294, filed on Sep. 2, 2015.

(51) Int. Cl. A61B 5/00; G01D 5/353

(52) U.S. Cl. CPC A61B 5/0084; G01D 5/35354; A61B 5/742

(57) ABSTRACT

A measurement probe includes: a plurality of optical fibers including an illumination fiber configured to propagate light to irradiate a measuring object and including a light receiving fiber configured to receive scattered light returned from the measuring object; and an optical member. The optical member is provided on an end portion of the measurement probe so as to face the measuring object, abuts on the plurality of optical fibers, and includes an exposed portion in which a part of a side portion of the optical member is exposed on an outside of the measurement probe, the side portion forming a surface of the optical member along a longitudinal direction of the measurement probe, the exposed portion being provided on an end portion of the optical member opposite to where the optical member abuts on the plurality of optical fibers.

### CROSS-REFERENCE TO RELATED APPLICATIONS

This application is based upon and claims the benefit of priority from U.S. Provisional Patent Application No. 62/213,294, filed on Sep. 2, 2015, the entire contents of which are incorporated herein by reference.

### BACKGROUND

1. Technical Field

The disclosure relates to a measurement probe and an optical measurement system for measuring optical characteristics of living tissue.

2. Related Art

In recent years, an optical measurement apparatus has been known which irradiates living tissue with illumination light and estimates characteristics of the living tissue based on a measurement value of detection light reflected or scattered from the living tissue. The optical measurement apparatus is used in combination with an endoscope which observes organs such as digestive organs. As an example of such optical measurement apparatus, an optical measurement apparatus has been proposed that uses low-coherence enhanced backscattering (LEBS) for detecting characteristics of living tissue by irradiating the living tissue with low-coherent white light with short spatial coherence length from an illumination fiber of a measurement probe, by detecting scattered light entered at different angles by using a plurality of light receiving fibers, and by measuring intensity distribution of the scattered light by using a spectroscope provided on each light receiving fiber (refer to JP 5049415 B2, for example).

### SUMMARY

In some embodiments, a measurement probe includes: a plurality of optical fibers including an illumination fiber configured to propagate light to irradiate a measuring object and including a light receiving fiber configured to receive scattered light returned from the measuring object; and an optical member that: is provided on an end portion of the measurement probe so as to face the measuring object; abuts on the plurality of optical fibers; and includes an exposed portion in which a part of a side portion of the optical member is exposed on an outside of the measurement probe, the side portion forming a surface of the optical member along a longitudinal direction of the measurement probe, the exposed portion being provided on an end portion of the optical member opposite to where the optical member abuts on the plurality of optical fibers.

In some embodiments, an optical measurement system includes: an optical measurement apparatus configured to perform an optical measurement on a measuring object to measure characteristics of the measuring object; and a measurement probe having a plurality of optical fibers including an illumination fiber configured to propagate light to irradiate the measuring object and including a light receiving fiber configured to receive scattered light returned from the measuring object. The measurement probe includes an optical member that: is provided on an end portion of the measurement probe to face the measuring object; abuts on the plurality of optical fibers; and includes an exposed portion in which a part of a side portion of the optical member is exposed on an outside of the measurement probe, the side portion forming a surface of the optical member along a longitudinal direction of the measurement probe, the exposed portion being provided on an end portion of the optical member opposite to where the optical member abuts on the plurality of optical fibers. The optical measurement apparatus includes a detecting unit configured to detect whether the exposed portion is covered with the measuring object when the measurement probe receives the scattered light, according to a detection value based on the scattered light received by the measurement probe.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is an external view illustrating a configuration of an optical measurement system according to a first embodiment of the present invention;

FIG. 2 is a block diagram schematically illustrating a functional configuration of the optical measurement system according to the first embodiment of the present invention;

FIG. 3 is a partial cross-sectional view schematically illustrating a configuration of a distal end portion of a measurement probe according to the first embodiment of the present invention;

FIG. 4 is an illustrative diagram showing contact between the distal end portion of the measurement probe according to the first embodiment of the present invention and living tissue;

FIG. 5 is an illustrative diagram showing an ideal contact state between the distal end portion of the measurement probe according to the first embodiment of the present invention and the living tissue;

FIG. 6 is an illustrative diagram showing a non-ideal contact state between the distal end portion of the measurement probe according to the first embodiment of the present invention and the living tissue;

FIG. 7 is an illustrative diagram showing the contact between the distal end portion of the measurement probe according to the first embodiment of the present invention and the living tissue, and showing propagation of light in the ideal contact state;

FIG. 8 is an illustrative diagram showing the contact between the distal end portion of the measurement probe according to the first embodiment of the present invention and the living tissue, and showing propagation of light in the non-ideal contact state;

FIG. 9 is a graphical illustration of the contact between the distal end portion of the measurement probe according to the first embodiment of the present invention and the living tissue, which shows a detection value detected by each light receiving fiber when the distal end portion of the measurement probe is in contact with the living tissue;

FIG. 10 is a schematic view when an optical measurement system according to the first embodiment of the present invention is used in an endoscope system;

FIG. 11 is a partial cross-sectional view schematically illustrating a configuration of a distal end portion of a measurement probe according to a modification of a first embodiment of the present invention;

FIG. 12 is an illustrative diagram showing contact between a distal end portion of a measurement probe and living tissue when an optical measurement system according to a second embodiment of the present invention is used in an endoscope system, and showing propagation of light in an ideal contact state in the endoscope system; and

FIG. 13 is a graphical illustration of intensity distribution of light detected when the distal end portion of the measurement probe according to the second embodiment of the present invention and the living tissue are in contact with each other.

### DETAILED DESCRIPTION

Modes for carrying out the present invention (hereinafter, referred to as “embodiment(s)”) will be hereinafter described with reference to the drawings. The same reference signs are used to designate the same elements throughout the drawings. The drawings are schematic, so that it is noted that relationship between thickness and width of each member, a ratio between the respective members and the like might be different from actual ones. Dimensional relationship and the ratio might be different between the drawings. The present invention is not limited by the embodiments.

### First Embodiment

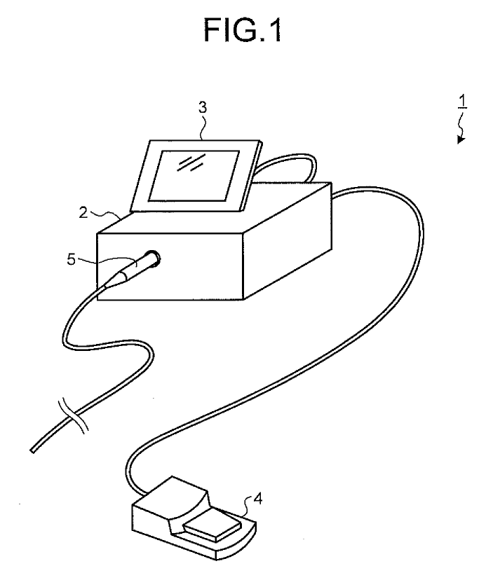

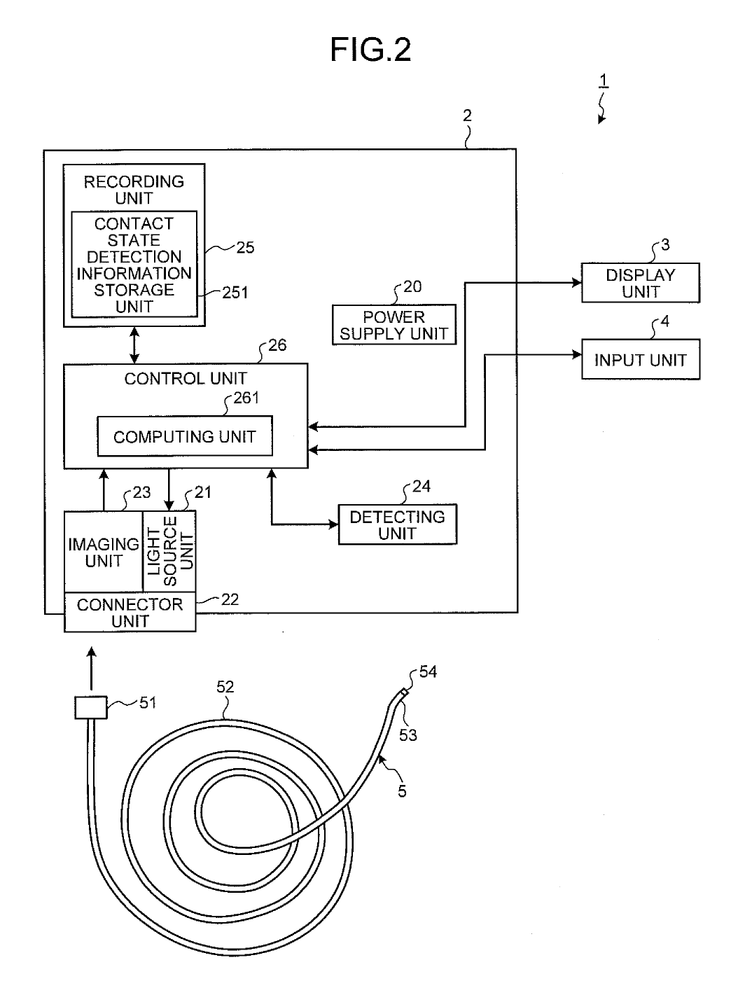

FIG. 1 is an external view illustrating a configuration of an optical measurement system 1 according to a first embodiment of the present invention. FIG. 2 is a block diagram schematically illustrating a functional configuration of the optical measurement system 1 according to the first embodiment of the present invention.

The optical measurement system 1 illustrated in FIGS. 1 and 2 includes an optical measurement apparatus 2 which performs optical measurement on a measuring object such as living tissue being a scatterer to detect characteristics (properties) of the measuring object, a display unit 3 which displays a measurement result of the optical measurement apparatus 2, an input unit 4 which accepts an input of an instruction signal to instruct the optical measurement apparatus 2 to measure, and an elongated measurement probe 5 detachably attached to the optical measurement apparatus 2 inserted into a subject.

A configuration of the optical measurement apparatus 2 will be first described. The optical measurement apparatus 2 includes a power supply unit 20, a light source unit 21, a connector unit 22, an imaging unit 23, a detecting unit 24, a recording unit 25, and a control unit 26. The power supply unit 20 supplies power to each unit of the optical measurement apparatus 2.

The light source unit 21 emits illumination light to the measurement probe 5 through the connector unit 22. The light source unit 21 is realized by using a light emitting element being an incoherent light source such as a white light emitting diode (LED), a condenser lens configured to collect light emitted by a light emitting element 21a, and a filter configured to pass light of a predetermined wavelength band, for example. The condenser lens, a collimating lens and the like may be used as such lens, for example. In the first embodiment, the light source unit 21 emits the illumination light including light of a component of a large numerical aperture (NA). The light source unit 21 emits incoherent light including at least one spectral component to the measurement probe 5 as the illumination light through the connector unit 22. The light emitting element may also be realized by using the incoherent light source such as a xenon lamp, a tungsten lamp, and a halogen lamp. One or more condenser lenses are provided as needed.

The connector unit 22 removably connects the measurement probe 5 to the optical measurement apparatus 2.

The imaging unit 23 receives return light of the illumination light emitted from a distal end of the measurement probe 5 and reflected and/or scattered from the measuring object, performs photoelectric conversion on the received light to generate an electric signal, and outputs the electric signal to the control unit 26. The imaging unit 23 is realized by using an image sensor such as a charge coupled device (CCD) image sensor, a complementary metal oxide semiconductor (CMOS) image sensor and the like.

The detecting unit 24 detects whether the distal end of the measurement probe 5 sinks into the measuring object based on intensity (detection value) of light of an image (spot) projected on a light receiving surface of the imaging unit 23 by the light emitted from each emission end of a plurality of optical fibers of the measurement probe 5.

The recording unit 25 records various programs for operating the optical measurement apparatus 2, various data and various parameters used by the optical measurement apparatus 2. The recording unit 25 is realized by using a volatile memory, a non-volatile memory and the like. The recording unit 25 temporarily records information and data being processed of the optical measurement apparatus 2. Furthermore, the recording unit 25 records the measurement result of the optical measurement apparatus 2 and arrangement information on arrangement of light receiving fibers. The recording unit 25 may also be formed of a removable memory card.

The recording unit 25 includes a contact state detection information storage unit 251 which stores information for detecting a contact state between the measurement probe 5 and the living tissue as a measuring object. Specifically, the contact state detection information storage unit 251 stores an ideal detection value detected in an ideal contact state and a threshold for determining the contact state, the threshold being based on a ratio (increase rate) of the detection value detected in a non-ideal contact state to the ideal detection value.

The control unit 26 controls processing operation of each unit of the optical measurement apparatus 2. The control unit 26 is formed of a central processing unit (CPU) and the like and performs overall control of the optical measurement apparatus 2 by transferring instruction information and data to each unit of the optical measurement apparatus 2. The control unit 26 includes a computing unit 261.

The computing unit 261 performs a plurality of pieces of arithmetic processing based on the electric signal input from the imaging unit 23 and calculates a characteristic value regarding the characteristics of the measuring object.

The display unit 3 outputs various pieces of information of the optical measurement apparatus 2. Specifically, the display unit 3 displays the information input from the optical measurement apparatus 2. The display unit 3 is realized by using a display panel of liquid crystal, organic electro luminescence (EL) or the like and a speaker. A touch panel for accepting input of a positional signal according to a contact position from outside may be provided on a display screen of the display unit 3.

The input unit 4 accepts the input of the instruction signal which instructs the optical measurement apparatus 2 to measure. The input unit 4 is realized by using an input interface such as a foot switch, a keyboard, and a mouse, for example.

The measurement probe 5 is formed of at least a plurality of optical fibers. Specifically, the measurement probe 5 is realized by using an illumination fiber (illumination channel) which emits the illumination light to the measuring object and a plurality of light receiving fibers (light receiving channels) which the return light of the illumination light reflected and/or scattered from the measuring object enters at different angles. The measurement probe 5 includes a proximal end portion 51 removably connected to the connector unit 22 of the optical measurement apparatus 2, a flexible portion 52 having flexibility, and a distal end portion 53 configured to emit the illumination light supplied from the light source unit 21 through the connector unit 22 and receives the return light of the illumination light from the measuring object. A rod lens 54 being an optical member coupled to the illumination fiber and the light receiving fiber on one end thereof to maintain a distance from the measuring object to the illumination fiber and the light receiving fiber constant is provided on a distal end of the distal end portion 53.

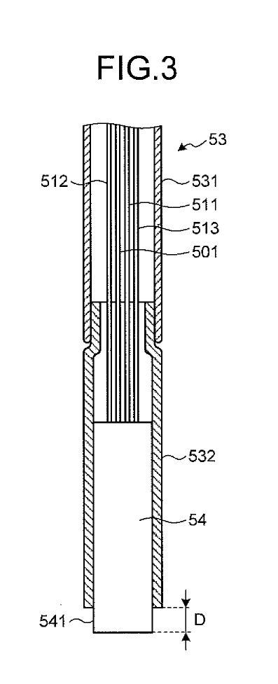

Here, reference will be made to a configuration of the distal end portion 53 of the measurement probe 5. FIG. 3 is a partial cross-sectional view schematically illustrating the configuration of the distal end portion 53 of the measurement probe 5. The partial cross-sectional view illustrated in FIG. 3 is a cross section taken along a plane passing through a central axis of the measurement probe 5 and is parallel to the central axis.

The measurement probe 5 includes an illumination fiber 501 which propagates the illumination light supplied from the light source unit 21 through the connector unit 22 to the distal end portion 53 of the measurement probe 5 to emit the illumination light to the measuring object and first, second, and third light receiving fibers 511, 512, and 513 which the return light of the illumination light reflected and/or scattered from the measuring object enters from the distal end portion 53 at different angles to propagate to the proximal end portion 51. Although there are three light receiving fibers in the first embodiment, the number thereof is not limited to three, and the light receiving fibers may be designed as needed.

The distal end portion 53 includes a tube 531 formed into a tube shape connected to the flexible portion 52 in which the illumination fiber 501 and the first, second, and third light receiving fibers 511, 512, and 513 are inserted, and a holding unit 532 formed into a tube shape attached to an end on a side different from a side of the flexible portion 52 of the tube 531 to hold the rod lens 54. The tube 531 and the holding unit 532 are formed of light blocking materials.

The holding unit 532 may be attached to the tube 531 by press fitting or may be attached by well-known bonding means (bonding by adhesive and the like) and fixing means (fitting, screwing and the like).

In the measurement probe 5, the illumination fiber 501 and the first, second, and third light receiving fibers 511, 512, and 513 abut on the rod lens 54. Therefore, the distance from the illumination fiber 501 and the first, second, and third light receiving fibers 511, 512, and 513 to the measuring object is maintained to length of the rod lens 54 in a longitudinal direction of the measurement probe 5.

The rod lens 54 includes an exposed portion 541. The exposed portion 541 is provided on an end portion of the rod lens 54 opposite to where the rod lens 54 faces the illumination fiber 501 and the first, second, and third light receiving fibers 511, 512, and 513, i.e., opposite to where the rod lens 54 abuts on the illumination fiber 501 and the first, second, and third light receiving fibers 511, 512, and 513. The rod lens 54 has a side portion forming a surface along the longitudinal direction of the measurement probe 5 (surface intersecting with a direction orthogonal to the longitudinal direction). A part of the side portion on the end portion of the rod lens 54 is exposed to form the exposed portion 541. In other words, at the distal end of the measurement probe 5, a part of the rod lens 54 projects from an end of the holding unit 532 to provide the exposed portion 541. A projecting amount (projecting length D illustrated in FIG. 3) in the longitudinal direction of the measurement probe 5 of the exposed portion 541 in the rod lens 54 may be the length enough to detect sinking of the measurement probe 5 into the measuring object; this is set to approximately few millimeters, for example.

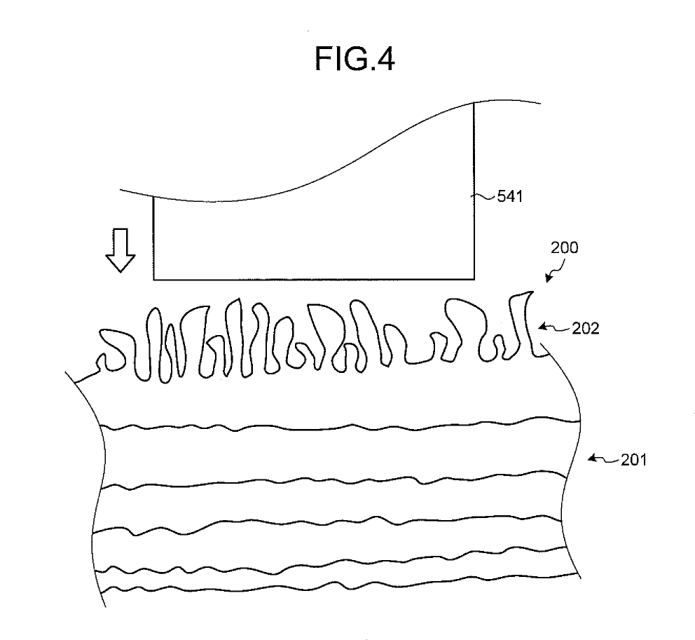

Next, contact between the measurement probe 5 and the living tissue being the measuring object well be described with reference to FIGS. 4 to 9. FIG. 4 is an illustrative diagram showing the contact between the distal end portion of the measurement probe according to the first embodiment of the present invention and the living tissue. Although the living tissue as the measuring object is a mucosa in the first embodiment, another tissue may be used. As illustrated in FIG. 4, living tissue 200 being the measuring object includes a submucosal layer 201 and a villus 202 being a tissue surface of the living tissue 200. At the time of measurement by the measurement probe 5, the distal end of the measurement probe 5 is brought into contact with the surface of the living tissue 200, the illumination fiber 501 irradiates the living tissue 200 with the illumination light through the rod lens 54, and the first, second, and third light receiving fibers 511, 512, and 513 receive the return light of the illumination light reflected and/or scattered from the living tissue 200 through the rod lens 54.

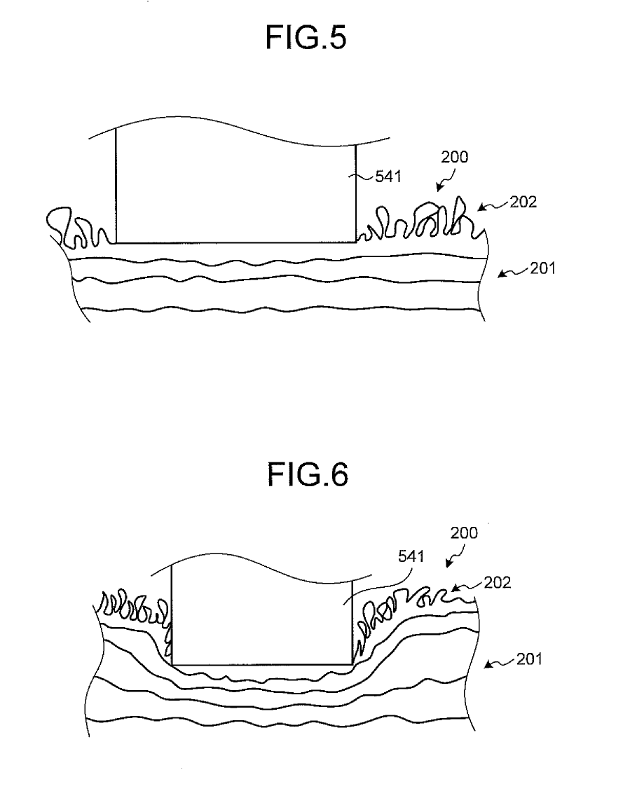

FIG. 5 is an illustrative diagram showing the ideal contact state between the distal end portion of the measurement probe according to the first embodiment of the present invention and the living tissue. FIG. 6 is an illustrative diagram showing the non-ideal contact state between the distal end portion of the measurement probe according to the first embodiment of the present invention and the living tissue. When the measurement probe 5 and the living tissue 200 are ideally in contact with each other, a distal end face of the rod lens 54 is in pressure contact with the villus 202 to crush the villus 202. At that time, a load from the measurement probe 5 is not applied to the submucosal layer 201 and the submucosal layer 201 is not deformed (refer to FIG. 5). On the other hand, when the measurement probe 5 and the living tissue 200 are non-ideally in contact with each other, for example, the distal end face of the rod lens 54 is in pressure contact with the villus 202 to crush the submucosal layer 201 and the villus 202, and the rod lens 54 sinks into the living tissue 200. In the non-ideal contact state, the load from the measurement probe 5 is applied also to the submucosal layer 201 and the submucosal layer 201 is deformed such that the exposed portion 541 is covered with the living tissue 200 (refer to FIG. 6).

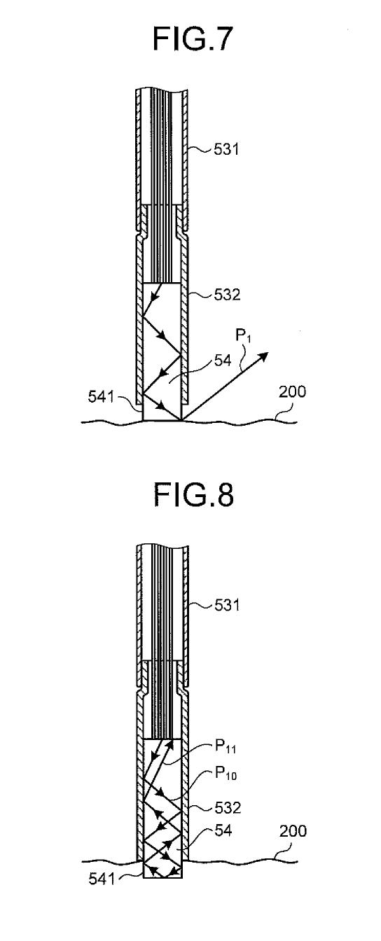

FIG. 7 is an illustrative diagram showing the contact between the distal end portion of the measurement probe according to the first embodiment of the present invention and the living tissue, and showing the propagation of light in the ideal contact state. FIG. 8 is an illustrative diagram showing the contact between the distal end portion of the measurement probe according to the first embodiment of the present invention and the living tissue, and showing the propagation of light in the non-ideal contact state. When the measurement probe 5 and the living tissue 200 are ideally in contact with each other (for example, refer to FIG. 5), the distal end face of the rod lens 54 is in pressure contact with the villus 202 to crush the villus 202. At that time, the side surface of the exposed portion 541 of the rod lens 54 is not in contact with the living tissue 200 and is exposed outside. Therefore, the illumination light emitted from the illumination fiber 501 is discharged outside from an exposed part of the exposed portion 541 (refer to FIG. 7). For example, as illustrated in FIG. 7, illumination light P₁ is discharged outside from the exposed part of the exposed portion 541. A trajectory of the illumination light P₁ illustrated in FIG. 7 is shown to be the same as that of illumination light with the highest NA.

On the other hand, when the measurement probe 5 and the living tissue 200 are non-ideally in contact with each other, i.e., when the rod lens 54 sinks into the living tissue 200, the side surface of the exposed portion 541 of the rod lens 54 enters the living tissue 200 to be covered with the villus 202 and the like. Therefore, the illumination light emitted from the illumination fiber 501 is reflected from the tissue surface of the living tissue 200 or reflected from an inner wall surface of the holding unit 532 to travel in the rod lens 54 without being discharged outside the exposed portion 541 (refer to FIG. 8). For example, as illustrated in FIG. 8, illumination light P₁₀ and illumination light P₁₁ are reflected from the tissue surface of the living tissue 200 or reflected from the inner wall surface of the holding unit 532 to travel in the rod lens 54. A trajectory of the illumination light P₁₀ and the illumination light P₁₁ illustrated in FIG. 8 is shown to be the same as that of illumination light with the highest NA.

As described above, when the measurement probe 5 and the living tissue 200 are ideally in contact with each other, a part of the illumination light is discharged outside, and when the measurement probe 5 and the living tissue 200 are non-ideally in contact with each other, the illumination light is not discharged outside and travels in the rod lens 54. Therefore, in the non-ideal contact state, the light receiving fiber receives the illumination light entering from the side surface of the exposed portion 541. When the light receiving fiber receives the illumination light entering from the side surface of the exposed portion 541, a measurement value in the light receiving fiber that is located farther from the illumination fiber to detect a high NA component significantly increases.

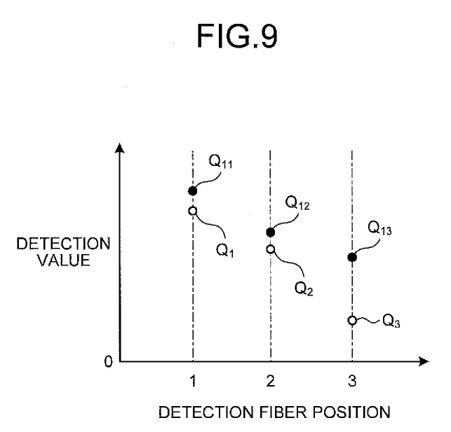

FIG. 9 is a graphical illustration of the contact between the distal end portion of the measurement probe according to the embodiment of the present invention and the living tissue, which shows the detection value detected by each light receiving fiber when the distal end portion of the measurement probe is in contact with the living tissue. In FIG. 9, the first, second, and third light receiving fibers 511, 512, and 513 are assigned with positional numbers of 1, 2, and 3 in the order of proximity to the illumination fiber 501. In FIG. 9, plots Q₁ to Q₃ indicated by white circles represent detection values detected in the ideal contact state. Plots Q₁₁ to Q₁₃ indicated by black circles represent detection values detected in the non-ideal contact state.

As illustrated in FIG. 9, the values of plots Q₁₁ to Q₁₃ are higher than the values of plots Q₁ to Q₃, respectively, in the detection values by all the light receiving fibers. Especially, an increase rate of the detection value by the third light receiving fiber the farthest from the illumination fiber 501 is significantly higher than that of the others. By using this phenomenon, it is possible to detect whether the exposed portion 541 sinks into the living tissue 200 and the reflection occurs.

Specifically, the detecting unit 24 refers to the detection value detected by the light receiving fiber and the ideal detection value detected in the ideal contact state stored in the contact state detection information storage unit 251, calculates a ratio of the detection value to the ideal detection value, and compares the ratio with the threshold, thereby detecting the contact state between the measurement probe 5 and the living tissue 200, in other words, detecting whether the exposed portion 541 sinks into the living tissue 200.

The control unit 26 may add information on the contact state to the characteristic value regarding the characteristics of the measuring object, or cause the display unit 3 to display the information on the detected contact state according to the detection result by the detecting unit 24. It is also possible to use light or sound to notify that the contact state is not ideal. If the light is used, a notifying unit is realized by using an LED, for example, and if the sound is used, the notifying unit is realized by using a buzzer and the like. According to this, it is possible to figure out whether the measurement is performed in the ideal contact state, i.e., in the contact state in which a highly reliable measurement result is obtained, or in the non-ideal contact state.

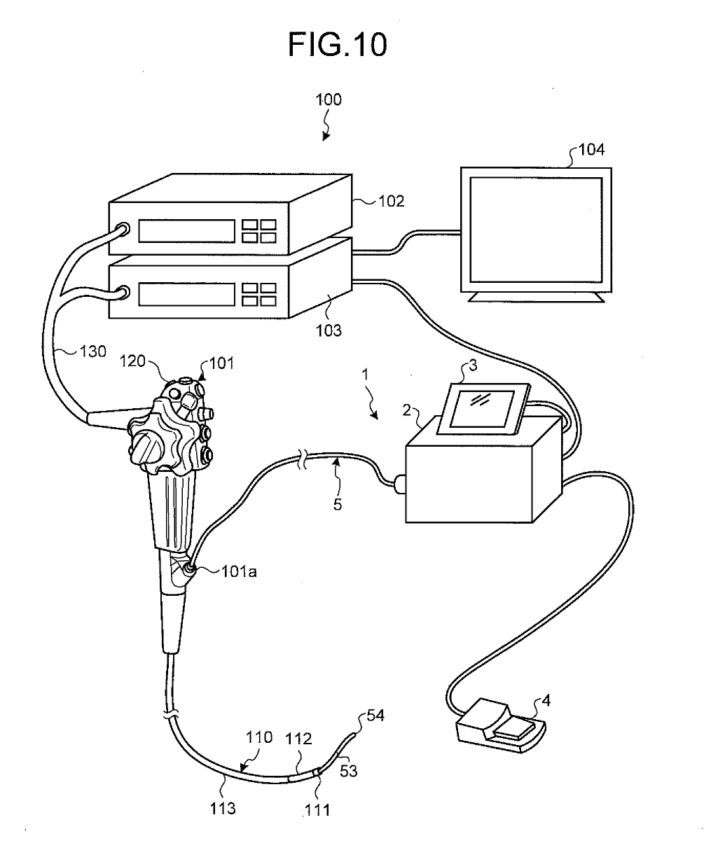

The optical measurement system 1 configured in the above-described manner may be used together with an endoscope 101 by inserting the measurement probe 5 through a treatment tool channel 101a provided on the endoscope 101 (endoscope scope) of an endoscope system 100 as illustrated in FIG. 10.

The endoscope system 100 includes the endoscope 101 configured to capture an in-vivo image of a subject by inserting a distal end portion into the subject, a light source device 102 configured to generate the illumination light to be emitted from a distal end of the endoscope 101, a processing device 103 configured to perform predetermined image processing on the in-vivo image captured by the endoscope 101 and to perform overall control of the entire endoscope system 100, and a display device 104 configured to display the in-vivo image on which the image processing has been performed by the processing device 103.

The endoscope 101 includes a flexible elongated insertion portion 110, an operating unit 120 connected to a proximal end side of the insertion portion 110 which accepts an input of various operating signals, and a universal code 130 extending from the operating unit 120 in a direction different from a direction in which the insertion portion 110 extends in which various cables connected to the light source device 102 and the processing device 103 are embedded.

The insertion portion 110 includes a distal end portion 111, a bendable portion 112, and an elongated flexible tube portion 113. The distal end portion 111 has therein an image sensor including pixels arranged two-dimensionally. Each pixel is configured to receive light and to perform photoelectric conversion on the light to generate a signal. The bendable portion 112 has a plurality of bending pieces. The flexible tube portion 113 is connected to a proximal end side of the bendable portion 112.

The measurement probe 5 is inserted into the endoscope 101 and inserted into the subject. The illumination fiber 501 emits the illumination light to the measuring object. Each of the first, second, and third light receiving fibers 511, 512, and 513 receives the return light of the illumination light reflected and/or scattered from the measuring object by the distal end portion 53 through the rod lens 54, and outputs the return light to the light receiving surface of the imaging unit 23. Thereafter, the computing unit 261 calculates the characteristic value of the characteristics of the measuring object based on information received by the imaging unit 23, and the detecting unit 24 detects the sinking of the measurement probe 5 into the measuring object. The control unit 26 outputs the detection result regarding the contact state between the measurement probe 5 and the living tissue by the detecting unit 24 together with an arithmetic result.

According to the first embodiment of the present invention described above, a part of the rod lens 54 which is brought into contact with the measuring object to form a transmission path of the light between the measuring object and the fiber is exposed outside from a distal end of the holding unit 532 at the distal end portion 53 of the measurement probe 5, so that it becomes possible to detect the sinking into the measuring object by using the characteristics of the detection value different between the case in which the measurement probe 5 sinks into the measuring object and the case in which this does not sink. According to this, it is possible to determine reliability of the obtained measurement result from the detection result of whether there is the sinking, and an appropriate measurement result may be obtained in the measurement performed by bringing the measurement probe into contact with the living tissue.

Modification of First Embodiment

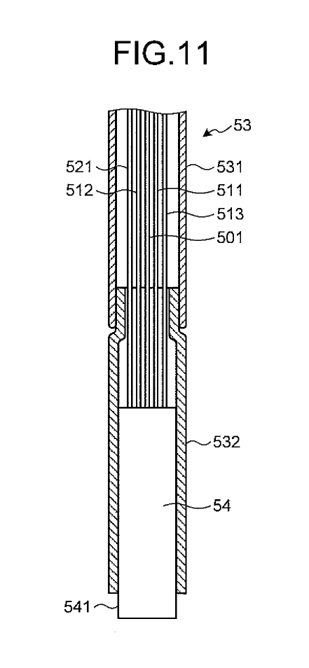

FIG. 11 is a partial cross-sectional view schematically illustrating a configuration of a distal end portion of a measurement probe according to a modification of a first embodiment of the present invention. The same reference signs are used to designate the same elements as those described above. In the above-described first embodiment, the sinking is detected by using the detection values detected by the three light receiving fibers. It is also possible to provide a detecting light receiving fiber 521 (second light receiving fiber) for receiving light having a high NA component in addition to the three light receiving fibers (first, second, and third light receiving fibers 511, 512, and 513) as in this modification. In this case, a detecting unit 24 determines whether a measurement probe 5 sinks into a measuring object by using a detection value detected by the detecting light receiving fiber 521.

In this modification, when the sinking occurs, it is possible to predict, from a length in a longitudinal direction of a rod lens 54, light with a high NA component that has been emitted from an illumination fiber 501 and has been reflected from living tissue to return to the detecting light receiving fiber 521, and it is also possible to predict an angle of the return light. Therefore, it is possible to obtain a length of an exposed portion 541 and arrangement of the detecting light receiving fiber 521 based on the length in the longitudinal direction of the rod lens 54.

### Second Embodiment

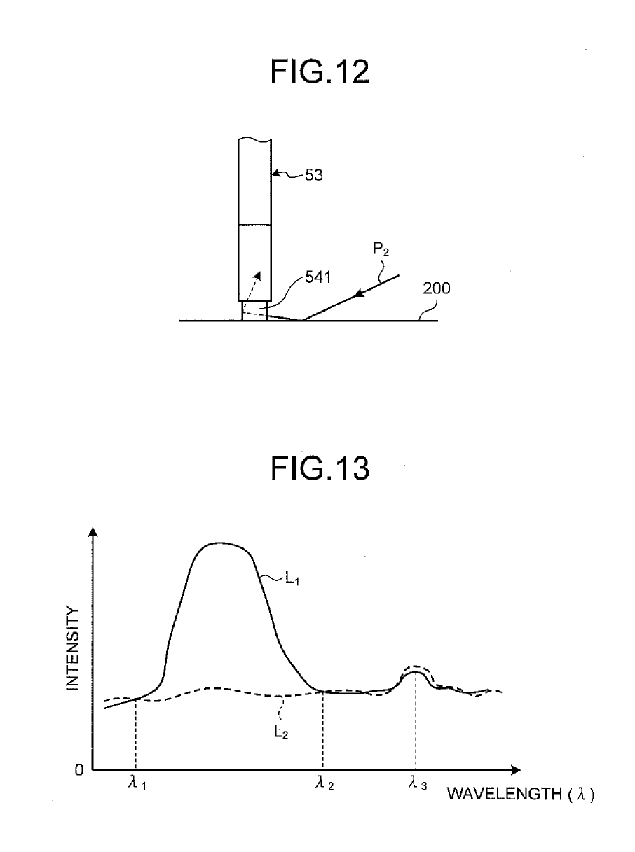

FIG. 12 is an illustrative diagram showing contact between a distal end portion of a measurement probe and living tissue when an optical measurement system according to a second embodiment of the present invention is used in an endoscope system, and showing propagation of light in an ideal contact state in the endoscope system. The same reference signs are used to designate the same elements as those described above. In the above-described first embodiment, the detecting unit 24 detects the sinking based on the detection value detected by receiving the return light of the illumination light by the light receiving fiber when the optical measurement system 1 is applied to the endoscope 101. In the second embodiment, however, the light receiving fiber receives the illumination light emitted from the endoscope 101, and the detecting unit 24 detects the sinking of a measurement probe 5 based on a light reception result.

In the second embodiment, a light source device 102 emits narrow band light including light of a partial wavelength band of a green wavelength band and light of a partial wavelength band of a blue wavelength band. Specifically, a partial wavelength band of the green wavelength band is a range not lower than 530 nm and not higher than 550 nm and a partial wavelength band of the blue wavelength band is a range not lower than 390 nm and not higher than 445 nm. Emission light limited to this band is referred to as narrow band illumination light and observation of an image by using the narrow band illumination light is referred to as a narrow band imaging (NBI) mode. It is possible to obtain an image in which a capillary vessel, mucosa micro pattern and the like present on a mucosa surface layer of a living body (living body surface layer) are enhanced by the narrow band imaging mode.

When the measurement probe 5 and living tissue 200 are ideally in contact with each other, as illustrated in FIG. 12, light P₂ that has been emitted from the light source device 102 and reflected from the living tissue 200 enters a rod lens 54 through a side surface of an exposed portion 541. The light P₂ entering the rod lens 54 from the side surface of the exposed portion 541 to travel while folding in the rod lens 54 enters a light receiving fiber (first light receiving fiber 511 and the like) thereafter. The light P₂ detected by the light receiving fiber enters an imaging unit 23 to be detected by the detecting unit 24.

On the other hand, when the measurement probe 5 and the living tissue 200 are non-ideally in contact with each other, the exposed portion 541 is covered with the living tissue 200 and the like as described above, so that the light P₂ does not enter the rod lens 54.

FIG. 13 is a graphical illustration of intensity distribution of the light detected when the distal end portion of the measurement probe according to the second embodiment and the living tissue are in contact with each other. The intensity distribution of the light illustrated in FIG. 13 illustrates intensity of the light with respect to a wavelength (λ) of the light. In FIG. 13, a solid curve L₁ indicates the intensity of the light detected in the ideal contact state and a broken curve L₂ indicates the intensity of the light detected in the non-ideal contact state.

As illustrated in FIG. 13, in the intensity distribution (curve L₁) when the measurement probe 5 and the living tissue 200 are ideally in contact with each other, the intensity increases at the wavelength according to the wavelength band of the light emitted from the light source device 102 and there is a peak in the above-described narrow band range. Therefore, when the measurement probe 5 and the living tissue 200 are ideally in contact with each other, the peaks are in the narrow band range (for example, a range from a wavelength λ₁ to a wavelength λ₂ illustrated in FIG. 13) and in a range of the wavelength band to be measured (central wavelength λ₃). The wavelength λ₁ is 390 nm, for example, and the wavelength λ₂ is 445 nm, for example.

In contrast, in the intensity distribution (curve L₂) when the measurement probe 5 and the living tissue 200 are non-ideally in contact with each other, there is no change in intensity at the wavelength according to the wavelength band of the light emitted from the light source device 102. Therefore, when the measurement probe 5 and the living tissue 200 are non-ideally in contact with each other, that is to say, when the measurement probe 5 sinks into the living tissue 200, the distribution has the peak only in the range of the wavelength band to be measured (central wavelength λ₃).

Since the intensity distribution changes according to the contact state between the measurement probe 5 and the living tissue 200 in this manner, the detecting unit 24 detects whether the measurement probe 5 and the living tissue 200 are ideally in contact with each other by confirming a peak position of the obtained intensity distribution. Specifically, the detecting unit 24 determines whether the intensity distribution of the light has the peak in the range according to the wavelength band of the light emitted by the light source device 102 and determines the contact state between the measurement probe 5 and the living tissue 200 by presence of the peak.

According to the second embodiment of the present invention described above, a part of the rod lens 54 which is brought into contact with the measuring object at a distal end portion 53 of the measurement probe 5 to form a transmission path of the light between the measuring object and the fiber is exposed outside from a distal end of a holding unit 532, so that it becomes possible to detect the sinking into the measuring object by using characteristics of the detection value different between the case in which the measurement probe 5 sinks into the measuring object and a case in which this does not sink. According to this, an appropriate measurement result may be obtained when performing the measurement while bringing the measurement probe into contact with the living tissue.

In the above-described second embodiment, the sinking of the measurement probe 5 is detected based on a light reception result of the illumination light emitted by the endoscope 101. It is also possible to detect the sinking based on the light reception result of the light emitted by the illumination device other than the endoscope 101 or to detect the sinking based on the light reception result of light such as natural light.

In the above-described first and second embodiments, the exposed portion 541 is provided such that the distal end of the rod lens 54 projects from the holding unit 532. The entire periphery may not be exposed and it is also possible to expose a part of the rod lens 54 by cutting a part of the holding unit 532 covering the side portion of the rod lens 54.

According to some embodiments, it is possible to obtain appropriate measurement results when performing a measurement while bringing a measurement probe into contact with the living tissue.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A measurement probe comprising:
a plurality of optical fibers including an illumination fiber configured to propagate light to irradiate a measuring object and including a light receiving fiber configured to receive scattered light returned from the measuring object; and
an optical member that:
is provided on an end portion of the measurement probe so as to face the measuring object;
abuts on the plurality of optical fibers; and
includes an exposed portion in which a part of a side portion of the optical member is exposed on an outside of the measurement probe, the side portion forming a surface of the optical member along a longitudinal direction of the measurement probe, the exposed portion being provided on an end portion of the optical member opposite to where the optical member abuts on the plurality of optical fibers.

2. The measurement probe according to claim 1, further comprising a second light receiving fiber configured to receive light having a component of a large numerical aperture out of light entering the measurement probe through the optical member.

3. An optical measurement system comprising:
an optical measurement apparatus configured to perform an optical measurement on a measuring object to measure characteristics of the measuring object; and
a measurement probe having a plurality of optical fibers including an illumination fiber configured to propagate light to irradiate the measuring object and including a light receiving fiber configured to receive scattered light returned from the measuring object, wherein
the measurement probe comprises an optical member that:
is provided on an end portion of the measurement probe to face the measuring object;
abuts on the plurality of optical fibers; and
includes an exposed portion in which a part of a side portion of the optical member is exposed on an outside of the measurement probe, the side portion forming a surface of the optical member along a longitudinal direction of the measurement probe, the exposed portion being provided on an end portion of the optical member opposite to where the optical member abuts on the plurality of optical fibers, and
the optical measurement apparatus comprises a detecting unit configured to detect whether the exposed portion is covered with the measuring object when the measurement probe receives the scattered light, according to a detection value based on the scattered light received by the measurement probe.

4. The optical measurement system according to claim 3, wherein the optical measurement apparatus further comprises a light source unit configured to output illumination light to the illumination fiber of the measurement probe, the illumination light having a component of a large numerical aperture.

5. The optical measurement system according to claim 3, further comprising a storage unit configured to store information for detecting a contact state between the measurement probe and the measuring object, wherein
the detecting unit is configured to detect the contact state between the measurement probe and the measuring object based on the detection value and the information stored in the storage unit.

6. The optical measurement system according to claim 3, further comprising a display unit configured to display information on characteristics of the measuring object measured based on a light reception result by the measurement probe, and to display a detection result by the detecting unit regarding a contact state between the measurement probe and the measuring object.

7. The optical measurement system according to claim 3, further comprising a notifying unit configured to notify of a detection result by the detecting unit regarding a contact state between the measurement probe and the measuring object.
